(19) United States
(12) Patent Application Publication
(10) Pub. No.: US 2006/0292607 A1
(43) Pub. Date: Dec. 28, 2006

(54) ANALYSIS OF TISSUE SAMPLES SURROUNDING MALIGNANCIES

(75) Inventor: RICHARD CAPRIOLI, Brentwood, TN (US)

Correspondence Address: FULBRIGHT & JAWORSKI L.L.P., 600 CONGRESS AVE., SUITE 2400, AUSTIN, TX, 78701, US

(73) Assignee: Vanderbilt University

(21) Appl. No.: 11/422,758

(22) Filed: Jun. 7, 2006

Related U.S. Application Data
(60) Provisional application No. 60/688,609, filed on Jun. 8, 2005.

(51) Int. Cl. C12Q 1/68; G01N 33/574; G06F 19/00

(52) U.S. Cl. 435/6; 435/7.23; 436/86; 702/19

(57) ABSTRACT

The present invention provides a method for analyzing tissue from the surgical margin of resected tumor, and the use of such information to predict recurrence, survival, and treatment efficacy in cancer patients. Methods of treatment based thereon are provided.

### BACKGROUND OF THE INVENTION

This application claims benefit of priority to U.S. Provisional Application Ser. No. 60/688,609, filed Jun. 8, 2005, the entire contents of which are hereby incorporated by reference.

### FIELD OF THE INVENTION

The present invention relates generally to the fields of molecular biology and oncology. More particularly, it concerns measurement of biological molecules in situ in a tissue sample to identify molecular defects in cells in the margins of resected tumors.

### BACKGROUND

All too often, the surgical removal of a tumor does not completely remove all cancerous cells from the patient. The risk of recurrence in the anatomical area from which the tumor was removed remains high. While some of these recurrences may be due to an incompletely removed tumor, in other cases the recurrence occurs in genetically altered cells in a field surrounding the area from which the tumor was excised (Tabor et al., 2001; Partidge et al., 2000). This field of genetically altered cells is known as a “field cancerization,” defined as a precancerous group of epithelial cells of monoclonal origin (Braakhuis et al., 2003). The term “field cancerization” was first introduced by Slaughter et al. (1953) who proposed that abnormal tissues surrounding oral squamous cell carcinoma was the source of subsequent primary tumors or locally recurrent cancers. Slaughter identified abnormal tissues by standard histopathologic techniques available in the 1950's. The methods to detect cellular aberrancies have become increasingly sophisticated and recent advances in molecular techniques have led to the identification of genetic and epigenetic defects in malignant and benign appearing cells alike (Braakhuis et al., 2003). Subsequently, field cancerization has been described in the tissues surrounding myriad primary cancers and have been attributed as significant factors for locoregional recurrences (Braakhuis et al., 2003).

Molecular and genetic data support a model that details the development of a field in which genetically altered cells play a central role in cancer recurrences. Initially, genetic transformation may focally occur in a group of cells forming a lesion of altered cells (Braakhuis et al., 2003). The expansion of this local field into a wider area underscores the critical steps in epithelial carcinogenesis (Braakhuis et al., 2003). Additional molecular/genetic alterations likely occur in a Vogel-gram-like progression (Cubilla et al., 1975), resulting in striking alterations in cell physiology that is representative of malignancy (Hruban et al., 2000). By virtue of its growth advantage, a proliferating field gradually displaces the normal mucosa. This concept is important for cancers of the pancreas, oral cavity, oropharynx, larynx, lung, vulva, esophagus, cervix, breast, skin, colon, and bladder (Braakhuis et al., 2003).

Alternatively, the molecular/genetic abnormalities in the histopathology negative tumor margins can represent tumor cells invading and metastasizing from the original primary tumor. The genetic defects that comprise the genotypic features of cancerous growths may coexist in benign appearing cells adjacent to the primary tumor and cannot be assessed intraoperatively by conventional histopathologic techniques. The implication is that cancerization fields may remain after surgery of the primary tumor and may subsequently develop into locoregionally recurrent cancers. Thus, the ability to detect field cancerization would greatly aid clinicians in the assessment and treatment of progressive cancer.

In most cases, field cancerization is impossible to visually distinguish from normal tissue. Thus, pre-cancerous growths may remain undetected until tumorigenesis occurs. Currently, methods of detecting field cancerization depend on molecular biology techniques such as fluorescence and in situ hybridization. These techniques are more successful at detecting field cancerization, but nevertheless there is an urgent need for better clinical detection techniques.

### SUMMARY OF THE INVENTION

In accordance with the present invention, there is provided a method for predicting recurrence of cancer, survival of cancer, and/or cancer progression in a cancer patient undergoing surgical resection comprising (a) obtaining a tissue sample from the surgical margin of the resected tumor; and (b) assessing histones in the tissue sample. Assessing a histone may be further defined as assessing a histone level and/or assessing a post-translational modification of a histone. Elevated histone levels are predictive of the recurrence of cancer, as are some post-translational modifications such as methylation or demethylation, alkyklation or dealkylation, and phosphorylation or dephosphorylation.

In certain embodiments, the invention provides a method for predicting field cancerization in a cancer patient comprising (a) obtaining a tissue sample from the surgical margin of the resected tumor; and (b) assessing histone levels in the tissue sample, wherein elevated histone levels in the tissue sample are predictive of field cancerization.

In other embodiments, the invention provides a method for predicting recurrence of cancer in a cancer patient undergoing surgical resection comprising (a) obtaining a tissue sample from the surgical margin of the resected tumor; and (b) assessing histone levels in the tissue sample, wherein elevated histone levels in the tissue sample are predictive of the recurrence of cancer.

In other embodiments, the invention provides a method for predicting survival of a cancer patient undergoing surgical resection comprising (a) obtaining a tissue sample from the surgical margin of the resected tumor; and (b) assessing histone levels in the tissue sample, wherein elevated histone levels in the tissue sample are predictive of the recurrence of cancer.

In other embodiments, the invention provides a method for predicting cancer progression in a cancer patient undergoing surgical resection comprising (a) obtaining a tissue sample from the surgical margin of the resected tumor; and (b) assessing histone levels in the tissue sample, wherein elevated histone levels in the tissue sample are predictive of the recurrence of cancer.

Those of ordinary skill in the art will be familiar with a variety of methods for assessing histone levels in tissue samples. In certain embodiments, assessing histone levels comprises (a) subjecting a spatially discrete microregion of an intact tissue sample to one or more physical or chemical treatments; and (b) assessing the histone levels in a protein sample from the microregion. If two or more spatially discrete microregions are assessed, then the histone levels of the different microregions may be compared. Assessing may also comprise immunologic detection of a histone or quantitative detection of an RNA (e.g., quantitative RT-PCR, Northern blotting) encoding a histone unit. Assessment of histones may comprise assessing a histone octamer, a linker histone, a histone unit, or a fragment thereof. The histone unit may be, for example H2A, H2B, H3, H4, H5, or H1.

An elevated histone level in a tissue refers to an amount of histone protein greater than the amount of histones in tissue of the same type that is neither cancerous nor pre-cancerous. The histone level in tissue that is neither cancerous nor pre-cancerous may be referred to as “normal histone level.”

In other embodiments, the invention provides a method for predicting recurrence of cancer in a cancer patient undergoing surgical resection comprising (a) obtaining a tissue sample from the surgical margin of the resected tumor; and (b) assessing histone post-translational modifications in the tissue sample, wherein altered histone post-translation modifications in the tissue sample are predictive of the recurrence of cancer.

In other embodiments, the invention provides a method for predicting survival of a cancer patient undergoing surgical resection comprising (a) obtaining a tissue sample from the surgical margin of the resected tumor; and (b) assessing histone post-translational modifications in the tissue sample, wherein altered histone post-translation modifications in the tissue sample are predictive of the recurrence of cancer.

In other embodiments, the invention provides a method for predicting cancer progression in a cancer patient undergoing surgical resection, comprising (a) obtaining a tissue sample from the surgical margin of the resected tumor; and (b) assessing histone post-translational modifications in the tissue sample, wherein altered histone post-translation modifications in the tissue sample are predictive of the recurrence of cancer.

Those of ordinary skill in the art will be familiar with a variety of methods for assessing histone post-translational modifications in tissue samples. In certain embodiments, assessing histone post-translational modifications comprise (a) subjecting a spatially discrete microregion of an intact tissue sample to one or more physical or chemical treatments; and (b) assessing the histone post-translational modifications in a protein sample from the microregion. If two or more spatially discrete microregions are assessed, then the histone post-translational modifications of the different microregions may be compared. Assessing may also comprise, for example, immunologic detection of post-translational modifications to a histone. Assessing may also comprise, for example, detection of histone modifications through the use of mass spectroscopy, high performance liquid chromatography, acrylamide gels, and any other laboratory technique related to protein biochemistry. Histone post-translational modifications may also be assessed indirectly, by assessing the level of expression or activity of enzymes that play a role in the post-translation modification of histones. Such enzymes include, for example, histone acetylases or deacetylases, histone methylases or demethylases, or histone kinases or phosphorylases.

Post-translational modifications may include acetylation, deacetylation, methylation, demethylation, phosphorylation, dephosphorylation, or ubiquitination. Altered histone post-translational modifications may be post-translational modifications to histones in cancerous or pre-cancerous tissue that are different from post-translational modifications to histones in normal (i.e., non-cancerous or non-precancerous) tissue of the same type.

In certain aspects of the invention, increased histone acetylation is predictive of cancer recurrence, cancer progression, and/or patient survival. In certain aspects of the invention, decreased histone acetylation is predictive of cancer recurrence, cancer progression, and/or patient survival. In certain aspects of the invention, increased histone methylation is predictive of cancer recurrence, cancer progression, and/or patient survival. In certain aspects of the invention, decreased histone methylation is predictive of cancer recurrence, cancer progression, and/or patient survival. In certain aspects of the invention, increased histone phosphorylation is predictive of cancer recurrence, cancer progression, and/or patient survival. In certain aspects of the invention, decreased histone phosphorylation is predictive of cancer recurrence, cancer progression, and/or patient survival. In certain aspects of the invention, increased histone ubiquitination is predictive of cancer recurrence, cancer progression, and/or patient survival. In certain aspects of the invention, decreased histone ubiquitination is predictive of cancer recurrence, cancer progression, and/or patient survival.

Analysis of histone levels and/or post-translational modifications may occur in situ, or may occur after removal of the protein sample from the microregion.

In another embodiment, there is provided a method predicting field cancerization in a cancer patient comprising (a) obtaining a tissue sample from the surgical margin of the resected tumor; (b) subjecting a spatially discrete microregion of the intact tissue sample to one or more physical or chemical treatments; and (c) analyzing a sample from the microregion for a cancer associated marker using mass spectrometry.

In another embodiment, there is provided a method predicting cancer progression in a cancer patient undergoing surgical resection of a tumor comprising (a) obtaining a tissue sample from the surgical margin of the resected tumor; (b) subjecting a spatially discrete microregion of the intact tissue sample to one or more physical or chemical treatments; and (c) analyzing a sample from the microregion for a cancer associated marker using mass spectrometry.

In another embodiment, there is provided a method predicting recurrence of cancer in a cancer patient undergoing surgical resection of a tumor comprising (a) obtaining a tissue sample from the surgical margin of the resected tumor; (b) subjecting a spatially discrete microregion of the intact tissue sample to one or more physical or chemical treatments; and (c) analyzing a sample from the microregion for a cancer associated marker using mass spectrometry.

In yet another embodiment, there is provided a method for predicting survival of a cancer patient undergoing surgical resection, comprising (a) obtaining a tissue sample from the surgical margin of the resected tumor; (b) subjecting a spatially discrete microregion of the intact tissue sample to one or more physical or chemical treatments; and (c) analyzing a tissue sample from the microregion for a cancer associated marker or markers using mass spectrometry. The number of markers may be 1, 2, 3, 4, 5 or more.

The methods of the present invention may fuirther comprise treating the cancer patient with an anti-cancer therapy, such as chemotherapy, radiotherapy, gene therapy, immunotherapy or hormonal therapy. The anti-cancer therapy may be customized based on the predicted risk of cancer recurrence or progression. For example, if a patient is determined to be at risk for cancer recurrence or progression, further treatment or more aggressive treatment for the cancer may be administered. Likewise, a patient identified a being at less risk for cancer recurrence or progression may not require additional anti-cancer therapy beyond surgical resection or may require a less aggressive treatment, thereby reducing unnecessary exposure to anti-cancer treatment. Moreover, the methods of the present invention may comprise modifying a patient's existing anti-cancer treatment based on an analysis of one or more of the methods herein described.

The one or more physical or chemical treatments may comprise solvent treatment, detergent treatment, lipase treatment, proteolysis, reactive agent treatment, snap freezing, sectioning with a cryostat, lyophilizing, or labeling. The labeling may comprise treatment with an isotope dilution reagent, a labeled antibody, or an enzyme. Two of the plurality of microregions may receive distinct physical or chemical treatments, or they may receive the same physical or chemical treatment.

Where the analysis occurs in situ, it may occur in one or more microregions (e.g., microwells) of an intact tissue sample. The microregion may be a microwell, optionally being between 5 and 200 microns, between 10 and 100 microns, or about 50 microns. The method may further comprise generating the microwell. Microwells may be created through the use of drills, lasers, or other physical means and/or chemical means such as acid, proteolysis, lipases, collagenases, detergents, or solvents that digest lipids or other material. In certain embodiments, the tissue sample comprises at least 6, 12, 18, 24, 30, 36, 42, 48, 54, 60, 66, 72, 78, 84, 90, 96, 102, 108, 114, or 120 microregions on a sample. In certain embodiments, the microregions will be created at a density of at least 1, 2, 4, 6, 8, 10, 12, 16, 20, or more microregions per square millimeter (mm²).

A cancer associated marker may be any molecule associated with a cancerous or pre-cancerous state. A molecule may be a cancer associated marker where, for example, its presence or absence, increased or decreased expression, post-translational modification, increased or decreased level, or subcellular localization are known to be associated with cancer, cancer cells, or pre-cancerous cells. The cancer associated markers may be, for example, proteins, nucleic acids, lipids, carbohydrates, drug metabolites, or any other biological molecule associated with the occurrence or progression of a cancer, neoplasia, or pre-neoplasia. Cancer associated markers may include acetylated histones, an elevated histone level, a larger amount of cell signaling tyrosine kinases (e.g., RAS, MAP-K) than would be found in non-cancerous tissue of the same tissue type, or a higher level of or differently modified growth receptors than would be found in non cancerous cells of the same tissue type. Any number of cancer associated markers may be analyzed. In certain embodiments, 1, 2, 3, 4, 5, 10, 15, 20, 25, or more cancer associated markers are analyzed.

A tumor may be from any type of tumor, for example, a lung tumor, esophageal tumor, brain tumor, pancreatic tumor, liver tumor, stomach tumor, intestinal tumor, rectal tumor, breast tumor, uterine tumor, cervical tumor, prostate tumor, testicular tumor, or skin tumor.

It is contemplated that any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.” It is contemplated that any embodiment discussed in this specification can be implemented with respect to any method or composition of the invention, and vice versa. Furthermore, compositions and kits of the invention can be used to achieve methods of the invention. Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D: In situ proteomics in tissue microwells. (FIG. 1A) Organic solvent is microprinted onto a mouse brain hemisphere resulting in tissue microwells of 150 82 m diameter. (FIG. 1B) Buffered solutions of trypsin are dispensed into the wells. The wells serve as an anchor and substrate for the reaction. (FIG. 1C) Magnified view of the tissue microwells after tryptic digestion. (FIG. 1D) MALDI TOF MS spectra obtained from a single tissue microwell after microdispensing of matrix solution into the wells.

### DETAILED DESCRIPTION OF ILLUSTRATIVE EMBODIMENTS

I. Field Cancerization

The high recurrence rate of some cancers may be explained by field cancerization. Although the term was originally developed in connection with oral cancer, field cancerization has since been detected in a number of organs (Boudewijn, 2003). One theory on the mechanism of field cancerization is that cells undergo pre-neoplastic, or pre-cancerous, changes that convey a growth advantage over surrounding normal tissue. Subsequent genetic mutations in these pre-neoplastic monoclonally derived fields can lead to one or more cells transforming into a full, invasive cancer. Another theory on the mechanism of field cancerization is that it results from migrated malignant or progenitor cells. Field cancerization is especially pernicious because it can escape standard histological assays. Thus, modern methods of detecting field cancerization draw on molecular genetics laboratory techniques.

However, a number of weaknesses remain in the detection of field cancerization by molecular genetic techniques. For example, all possible genetic mutations leading to transformation have yet to be characterized. This means that a mutation could escape genetic analysis. Moreover, many tests focus exclusively on mutations in the p53 tumor suppressor gene, but not all cancers feature mutations to this gene. Because of the significant problem field cancerization poses to human health, there is an urgent need for faster, more comprehensive methods of detecting neoplastic and/or pre-neoplastic changes in the tissue surrounding a resected tumor. The present invention provides novel methods for the detection of field cancerization.

II. Detecting Field Cancerization with Mass Spectrometry

One embodiment of the present invention is a method for detecting field cancerization in a cancer patient undergoing surgical resection by examining tissue from the surgical margin of a resected tumor for cancer associated markers using mass spectrometry. A cancer associated marker may be any molecule associated with a cancerous or pre-cancerous state. A molecule may be a cancer associated marker where, for example, its presence or absence, increased or decreased expression, post-translational modification, increased or decreased level, or subcellular localization are known to be associated with cancer, cancer cells, or pre-cancerous cells. The cancer associated markers may be, for example, proteins, nucleic acids, lipids, carbohydrates, drug metabolites, or any other biological molecule associated with the occurrence or progression of a cancer, neoplasia, or pre-neoplasia. Cancer associated markers may include acetylated histones, an elevated histone level, a larger amount of cell signaling tyrosine kinases (e.g., RAS, MAP-K) than would be found in non-cancerous tissue of the same tissue type, or a higher level of or differently modified growth receptors than would be found in non cancerous cells of the same tissue type. The presence of field cancerization in a cancer patient may be associated with poor clinical outcome. For example, field cancerization can be predictive of cancer recurrence, cancer progression, and/or decreased survival time. The ability to detect field cancerization according to the present invention will enable better treatment of cancer patients.

A. Mass Spectrometry Techniques

Mass spectrometry (MS) is a powerful method for detecting field cancerization by assaying any number of cancer associated markers. By exploiting the intrinsic properties of mass and charge, mass spectrometry (MS) can resolve and confidently identify a wide variety of complex compounds. Mass spectrometry is an excellent tool for identifying histones and histone modifications (Varreault et al., 2004).

There are a variety of mass spectrometry techniques known in the art. Traditional quantitative MS has used electrospray ionization (ESI) followed by tandem MS (MS/MS) (Chen et al., 2001; Zhong et al., 2001; Wu et al., 2000). Of particular interest in the present invention is matrix assisted laser desorption/ionization time of flight (MALDI-TOF) MS (Bucknall et al., 2002; Mirgorodskaya et al, 2000; Gobom et al., 2000). Another MS technique of interest in the present invention is surface enhanced laser desorption/ionization time of flight (SELDI-TOF) MS, which is a variation on MALDI-TOF. While MALDI-TOF and SELDI-TOF are preferred MS techniques, the present invention is not limited to a particular type of MS.

Traditional methods of examining proteomes with MS involve homogenizing small samples of tissues, using separative techniques such as gel electrophoresis or liquid chromatography, which are followed by MS for detection. Although this method gives adequate results, it is tedious, labor intensive and destroys any spatial fidelity in the sample due to the homogenization process. Therefore, current approaches to MS quantification of protein expression require substantial improvements in sample processing and utilization.

In one embodiment, the present invention provides a method for analyzing proteins or other molecules in situ, i.e., directly in intact tissues and within discrete areas thereof. In particular embodiments, micron-sized wells are created in intact tissues of interest. The wells create “vessels” in which chemistries can be performed, such as detergent extractions, labeling reactions, etc. Subsequently, the wells can be interrogated with various techniques, particularly mass spectroscopy.

The present invention is not limited, however, to proteomics applications. With relative ease, the methods described herein may be advantageously applied to examining the lipid, carbohydrate, nucleic acid, metabolite or even drug content of a tissue.

1. ESI

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 82 L/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer, is delivered to tip through an internal passage from a suitable source (such as from a chromatograph or directly from a sample solution via a liquid flow controller). By applying pressure to the sample in the capillary, the liquid leaves the capillary tip as a small highly electrically charged droplets and further undergoes desolvation and breakdown to form single or multicharged gas phase ions in the form of an ion beam. The ions are then collected by the grounded (or negatively charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; U.S. Pat. No. RE 35,413; 6,756,586, 5,572,023 and 5,986,258.

2. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al, 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances sample clean up is required (Nelson et al., 1994; Gobom et al., 2000). Desorption electrospray is a new associated technique for sample surface analysis.

3. SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., 0, Cs), neutrals or even photons, forcing atomic and molecular particles to be ejected from the surface, a process called sputtering.

Since some of these sputtered particles carry a charge, a mass spectrometer can be used to measure their mass and charge. Continued sputtering permits measuring of the exposed elements as material is removed. This in turn permits one to construct elemental depth profiles.

4. LD-MS and LDLPMS

Laser desorption mass spectroscopy (LD-MS) involves the use of a pulsed laser, which induces desorption of sample material from a sample site-effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments are due to different size causing different velocity.

Each ion mass will thus have a different flight-time to the detector.

One can either form positive ions or negative ions for analysis. Positive ions are made from regular direct photoionization, but negative ion formation require a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

5. MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Zaluzec et al., 1995; Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al, 1997; Chaurand et al., 1999; Jespersen et al., 1999).

The properties that make MALDI-TOF-MS a popular qualitative tool-its ability to analyze molecules across an extensive mass range, high sensitivity, minimal sample preparation and rapid analysis times-also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al, 1998; Horak et al., 2001; Gobom et al., 2000; Wang et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al, 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al, 1996; Nelson et al, 1994; Duncan et al., 1993; Gobom et al., 2000; Wu et al., 1997; Mirgorodskaya et al, 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

Identification of proteins corresponding to predictive MALDI-TOF signals typically involves two approaches. First, protein extracts from tissue samples will be fractionated by HPLC, 1D-SDS-PAGE or solution phase isoelectric focusing and fractions exhibiting the MALDI-TOF MS signals of interest will be subjected to tryptic digestion and analysis by LC-MS-MS. Peptides and their corresponding proteins of origin are identified from MS-MS spectra with Sequest, which correlates uninterpreted MS-MS spectra with theoretical spectra from database sequences (Eng et al., 1994). Confirmation of the protein identities is based on apparent molecular weight of the MS-MS identified proteins compared to pattern-specific signals detected in the MALDI profiles.

A second identification approach will pair LC-MS-MS analyses with stable isotope tags. Protein extracts from two samples to be compared (e.g., samples that differ in MALDI proteome patterns) are chemically tagged with light and heavy (unlabeled vs. deuterium or ¹³C-labeled) reagents, then combined, digested and the tagged peptides are then analyzed by LC-MS-MS. Peptides derived from the two samples are distinguished by pairs of signals in full scan MS separated by the mass difference of the light and heavy isotope tags. Pairs of signals whose intensities deviate from unity represent proteins that were differentially present in the original two samples. MS-MS spectra acquired from these peaks in the same LC-MS-MS analyses allow unambiguous identification of the differentially expressed proteins. The best-known version of this approach uses the thiol-reactive ICAT reagents developed by Gygi and Aebersold (Gygi et al., 1999), although newer, acid-cleavable reagents offer more efficient recovery of tagged peptides and produce higher quality MS-MS spectra for identification (Zhou et al., 2002). N-terminal isotope tagging of tryptic peptides enables identification of proteins that differ in posttranslational modifications rather that protein expression level per se (Mason and Liebler, 2003).

6. SELDI-TOF MS

Surface-enhanced laser desorption ionization-time of flight mass spectrometry (SELDI-TOF MS) is a variant of MALDI-TOF mass spectrometry. In SELDI-TOF MS, fractionation based on protein affinity properties is used to reduce sample complexity. For example, hydrophobic, hydrophilic, anion exchange, cation exchange, and immobilized-metal affinity surfaces can be used to fractionate a sample. The proteins that selectively bind to a surface are then irradiated with a laser. The laser desorbs the adherent proteins, causing them to be launched as ions. The “time of flight” of the ion before detection by an electrode is a measure of the mass-to-charge ration (m/z) of the ion.

III. Tissue Microregions

A. Obtaining Tissue Specimens

In accordance with some embodiments of the present invention, tissue samples are obtained from the margins of resected tumors by standard methodologies. The tissue samples are preferably of a sufficient size to permit creation of a plurality of microregions, e.g., at least 1 micron to several millimeters, including sizes in between, such as 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 60 μm, 70 μm, 80 μm, 90 μm, 100 μm, 125 μm, 150 μm, 175 μm, 200 μm, 250 μm, 275 μm, 300 μm, 400 μm, 450 μm, 500 μm, 600 μm, 700 μm, 800 μm, 900 μm, 1000 μm, 2 mm, 3 mm, 4 mm and 5 mm. Biopsies, including fine needle aspirates, are specifically contemplated.

The tissues should be handled such that (a) the integrity of the tissue is maintained and (b) that the cells within the tissue, particularly those in the region(s) where microwells will be created, are not damaged. Appropriate physiologic buffers are generally applied to the tissue, or the tissues are immersed therein. The tissue may also be cooled to appropriate temperatures for limited periods of time. Steps should be taken to ensure that apoptosis or other cellular degradation will not be induced in the tissue specimen. In certain aspects of the invention, the tissues are snap frozen and sectioned in a cryostat for mounting on a slide.

B. Microregions

A microregion is an area on the tissue sample that comprise substructure or areas of cellular change or areas of unique interest because of their morphology. The size ranges from nanometers to millimeters. Microregions may be made chemically by solvents that act of lipids or connective proteins. Microregions may also be made physically, such as by a blunt object for tapping, or a sharp object for cutting, or a laser. It is preferable to avoid losing material.

IV. Sample Preparation

Once one has obtained and prepared tissues sections containing microwells according to the present invention, it will be necessary to treat the microwells in order to liberate proteins for further analysis. A wide variety of techniques may be applied to the microwells including detergent extraction, treatment with various enzymes (lipases, collagenases, proteases, nucleases) or even with enzyme inhibitors (protease inhibitors). Examples of these treatments are provided below.

A. Detergent Extraction

In order to perform mass spectroscopic or other analysis of protein materials from samples of the present invention, certain treatments are required to prepare the proteins. At a minimum, proteins will be solubilized using detergent extraction. A variety of detergents are available for protein extraction, including anionic, cationic, zwitterionic, and non-ionic detergents. By virtue of their amphipathic nature, detergents are able to disrupt bipolar membranes to first free and then solublize proteins bound in the membrane or found inside the target cells.

In selecting a detergent, consideration will be given to the nature of the target protein(s), and the fact that anionic and cationic detergents are likely to have a greater effect on protein structure than zwitterionic or non-ionic detergents. However, non-ionic detergents tend to interfere with charge-bases analyses like mass spectroscopy, and are also susceptible to pH and ionic strength. Zwitterionic detergents provide intermediate properties that, in some respects, are superior to the other three detergent types. Offering the low-denaturing and net-zero charge characteristics of non-ionic detergents, zwitterionics also efficiently disrupt protein aggregation without the accompanying drawbacks. Exemplary anionic detergents include chenodeoxycholic acid, N-lauroylsarconsine sodium salt, lithium dodecyl sulfate, 1-octanesulfonic acid sodium salt, sodium cholate hydrate, sodium deoxycholate, sodium dodecyl sulfate and glycodeoxycholic acid sodium salt. Cationic detergents include cetylpyridinium chloride monohydrate and hexadecyltrimethylammonium bromide. Zwitterionic detergents include CHAPS, CHAPSO, SB3-10 and SB3-12. Non-ionic detergents may be selected from N-decanoyl-N-methylglucamine, digitonin, n-dodecyl P-D-maltoside, octyl α-D-glucopyranoside, Triton X-100, Triton X-114, Tween20 and Tween80.

In certain aspects of the invention, a cleavable detergent, 3-[3-(1,1-bisalkyloxyethyl) pyridin- 1 -yl]propane- 1 -sulfonate (PPS), may be utilized. This detergent can be used to extract protein contained within the interior of the cell by disrupting cell membranes. Once the proteins are free from the cell, PPS also assists in protein solubilization by shielding the hydrophobic regions of the newly extracted protein from unfavorable interactions with water. The added advantage of PPS over conventional detergents such as sodium dodecyl sulfate or n-octylglucoside is that the detergent properties that interfere with MALDI mass spectrometry can be eliminated prior to analysis. PPS was found to improve sensitivity in MALDI analyses of both soluble proteins and membrane proteins without degrading spectral quality. The virtues of this strategy were applied to whole cell extracts (Norris et al., 2003).

B. Lipases

To the extent that lipid removal by detergent extraction is incomplete, one may choose to utilize enzymes to further degrade lipid contaminants. Such enzymes are called lipases, almost all of which exhibit the catalytic triad Ser-Asp-His (an exception being geotrichium candidumm, which has Ser-Glu-His). The areas of the protein predicted to be involved in interfacial activation and conformational change show varying sequences, but a large number possess some sort of flap covering the active site.

Lipases are also characterized by the phenomena of interfacial activation. At very low substrate concentrations in aqueous solution, the enzymes are inactive. When the substrate concentration is high enough to form lipid micelles, the enzyme becomes activated. Though the mechanism in not yet fully understood, it appears that the lipase exists in two (or perhaps more) possible conformations. In one extreme, there is a conformation in which a structural element covers the active site, while at the other extreme, there is a structure in which the active site is exposed, allowing substrate to gain access. The micelle may initiate exposure of the active site.

Lipases have been isolated from a wide variety of mammalian and microbial sources. The mammalian lipases can be split into four groups, the hepatic lingual, gastric and pancreatic lipase and microbial lipases into bacterial and fungal. Very little homology has been found within the known sequences, the most conserved feature being the consensus sequence GxSxG found in the substrate binding site. The above-mentioned catalytic triad (Ser-Asp-His) is also a highly conserved. However, this is common to all esterases, not just lipases, as is the c/phydrolase fold.

Known lipases include triacylglycerol lipase (triglyceride lipase; tributyrase) phospholipase A2 (phosphatidylcholine 2-acylhydrolase, lecithinase A, phosphatidase, phosphatidolipase) lysophospholipase (lecithinase B, lysolecithinase, phospholipase B) acylglycerol lipase (monoacylglycerol lipase) galactolipase, phospholipase Al, lipoprotein lipase (clearing factor lipase, diglyceride lipase, diacylglycerol lipase) dihydrocoumarin lipase, 2-acetyl-1-alkylglycerophosphocholine esterase (1-alkyl-2-acetylglycerophosphocholine esterase, platelet-activating factor acetylhydrolase, PAF acetylhydrolase, PAF 2-acylhydrolase, LDL-associated phospholipase A2 LDL-PLA(2)), phosphatidylinositol deacylase (phosphatidylinositol phospholipase A2) phospholipase C (lipophosphodiesterase I, Lecithinase C, Clostridium welchii α-toxin, Clostridium oedematiens β- and γ-toxins) phospholipase D, (lipophosphodiesterase II, lecithinase D, choline phosphatase), phosphoinositide phospholipase C (triphosphoinositide phosphodiesterase, phosphoinositidase C, 1-phosphatidylinositol-4,5-bisphosphate phosphodiesterase, monophosphatidylinositol phosphodiesterase. phosphatidylinositol phospholipase C, PI-PLC, 1-phosphatidyl-D-myo-inositol-4,5-bisphosphate inositoltrisphosphohydrolase), alkylglycerophosphoethanolamine phosphodiesterase. (lysophospholipase D), glycosylphosphatidylinositol phospholipase D (GPI-PLD, glycoprotein phospholipase D, phosphatidylinositol phospholipase D, phosphatidylinositol-specific phospholipase D, phosphatidylinositol-glycan-specific phospholipase D), phosphatidylinositol diacylglycerol-lyase (1-phosphatidylinositol phosphodiesterase, monophosphatidylinositol phosphodiesterase, phosphatidylinositol phospholipase C, 1-phosphatidyl-D-myo-inositol inositolphosphohydrolase (cyclic-phosphate-forming)), glycosylphosphatidylinositol diacylglycerol-lyase ((glycosyl)phosphatidylinositol-specific phospholipase C, GPI-PLC, GPI-specific phospholipase C, VSG-lipase, glycosyl inositol phospholipid anchor-hydrolyzing enzyme, glycosylphosphatidylinositol-phospholipase C, glycosylphosphatidylinositol-specific phospholipase C, variant-surface-glycoprotein phospholipase C).

C. Collagenases

Collagen is the most abundant protein in vertebrates, and occurs in almost every tissue. However, for many applications, is it necessary to remove collagen in order to analyze other proteins in a sample. Moreover, analysis of collagen may be of only limited interest. As a result, methods for the removal of collagen are regularly employed in tissue dissociation.

Collagenases are enzymes that are able to cleave the peptide bonds in triple helical collagen molecules. Collagenases from Clostridium histolyticum have been known and studied for decades. Clostridopeptidase and clostripain activities also are associated with some collagenase preparations. Collagenase has been shown effective in isolating intact cells from a variety of tissues including bone, cartilage, thyroid, ovary, uterus, skin, endothelium, neuronal, pancreas, heart, liver and tumors.

D. Nucleic Acid Removal

Elimination of nucleic acids from sample prior to analysis can be achieved by chemical or enzymatic means. Chemical removal involves precipitation methods that employ polyethyleneimine (PEI) or streptomycin sulfate precipitation, followed by centrifugation.

Alternatively, enzymes that specifically degrade DNA and/or RNA may be used to remove these molecules. Benzonase is a genetically engineered endonuclease from Serratia marcescens. The protein is a dimer of two 30 kDa subunits. The enzyme degrades all forms of DNA and RNA, including single-stranded, double-stranded, linear and circular molecules, and is effective over a wide range of operating conditions. Some sequence specificity has been identified, with GC-rich regions being preferred. More selective enzymes that degrade DNA (DNases) or RNA (RNases) can be utilized as well.

E. Buffers

Once extracted, buffers will often be utilized to preserve the integrity of protein samples. Buffers are aqueous solutions composed of a weak acid (proton donor) and its conjugate base (proton acceptor). The acid or base is partially neutralized and shows little pH change in response to the addition of stronger acids or bases because of the buffers ability to “absorb” hydrogen ions, which determine pH. The most effective pH range for a buffer is generally one pH unit and is centered around the pKₐ of the system.

In choosing an appropriate buffer system, one generally takes into account the following considerations. (1) The pKa of the buffer should be near the desired midpoint pH of the solution. (2) The capacity of the buffer should fall within one to two pH units above or below the desired pH values. If the pH is expected to drop during the procedure, one should choose a buffer with a pKₐ slightly lower than the midpoint pH. Similarly, if the pH is expected to rise, one should choose a buffer with a slightly elevated pH. (3) The concentration of the buffer should be adjusted to have enough capacity for the experimental system. (4) The pH of the buffer should be checked at the temperature and concentration which will be used in the experimental system. (5) No more than 50% of the buffer components should be dissociated or neutralized by ionic constituents which are generated within or added to the solution. (6) Buffer materials should not absorb light between the wavelengths of 240-700 nm.

Useful buffers include ADA (Na salt), BES, ethyl glycinate, glycine, PBS, lithium citrate, PIPPS, potassium phosphate (mono- or dibasic), sodium citrate, sodium phosphate, TAPS, Tris base, Tris-HCl, MES, Bis-Tris, PIPES (Na salt), ACES, MOPES, TES, HEPES (Na salt), HEPPS, Tricine, Bicine, CHES, CAPS, MOPSO, DIPSO, HEPPSO, POPSO, AMPSO and CAPSO.

Particularly useful buffers for mass spectrometry are volatile buffers, including ammonium bicarbonate and ammonium acetate.

F. Protease Inhibitors

In order to prevent proteins from being non-specifically degraded following extraction, it may be necessary to include inhibitors of proteases, which are enzymes that hydrolyze peptide bonds. Proteases or peptidases are usually categorized as endopeptidases, which cleave internal bonds, or exopeptides, which remove residues from the termini of protein chains. Alternatively, proteases may be classified by virtue of their target sites, such as serine or cysteine residues.

The following protease-selective inhibitors are known to be useful in accordance with the present invention: antipain dihydrochloride (papain, trypsin); calpain inhibitor I (calpain I and II); calpain inhibitor II (calpain II and I); chymostatin (chymotrypsin); hirudin (thrombin); TLCK-HC1 (trypsin, bromelain, ficin, papain); TPCK (chymotrypsin, bromelain, ficin, papain); and trypsin inhibitor (trypsin). Other inhibitors include APMSF, aprotinin, bestatin, leupeptin, pepstatin, PMSF and TIMP-2.

G. Proteolysis

In other embodiments, it may be desirable to fragment peptides, albeit in a controlled fashion. There are two basic methods for digesting proteins: enzymatic and chemical methods. Enzymatic digestions are more common. An ideal digestion cuts only at a specific amino acid, but cuts at all occurrences of that amino acid. The number of digestion sites should not produce too many peptides because separation of peptides becomes too difficult. On the other can, too few digestions produces peptides too large for certain kinds of analysis.

The most common digestions are with trypsin and with lysine specific proteinases, because these enzymes are reliable, specific, and produce a suitable number of peptides. The next most common digestion is at aspartate or glutamate using endoproteinase Glu-C or endoproteinase Asp-N. Chymotrypsin is sometimes used, although it does not have a well defined specificity. Proteinases of broad specificity may generate many peptides, and the peptides may be very short. Of the chemical cleavages, cyanogen bromide is the most common. All the chemical digestions are less efficient than a good enzymatic digest. However they do produce only a few peptides, which can ease any purification problem.

V. Histones

In one embodiment, the present invention provides a method for predicting recurrence of cancer, survival of cancer, and/or cancer progression in a cancer patient undergoing surgical resection, comprising (a) obtaining a tissue sample from the surgical margin of the resected tumor; and (b) assessing histones in the tissue sample. Assessing a histone may be further defined as assessing a histone level and/or assessing a post-translational modification of a histone. Elevated histone levels are predictive of the recurrence and/or progression of cancer, as are some post-translational modifications such as methylation or demethylation, alkyklation or dealkylation, and phosphorylation or dephosphorylation.

Histones are involved in condensing and decondensing DNA to or from chromatin. Within the structure of chromatin, 147 base pairs of DNA are wrapped in 1.7 left-handed superhelical turns around an octamer of histones, two each of histone subtypes H2A, H2B, H3, and H4 (Verreault, 2000). In the center of this mass is histone H1, which along with H5 is known as a linker histone. Linker histones are crucial in creating and maintaining the higher order structure of chromatin fiber, and also play a role in the transcriptional regulation of specific genes such as 5S rRNA (reviewed in Zlatanova et al., 2000).

Histones are of importance to cells because of their role in condensing and decondensing DNA to or from chromatin. Aside from preparing genetic material for events such as mitosis and apoptosis, the degree to which DNA is condensed has obvious physical implications for any biological process which uses DNA, especially transcription. Condensed DNA is less accessible to cellular transcription machinery. The degree to which DNA is incorporated into chromatin depends partially on ATP-dependent mechanisms within the cell, but also on post-translational modifications to histones. These modifications include serine/threonine phosphorylation, lysine acetylation, lysine and arginine methylation, and lysine ubiquitination.

The most well-studied class of modification is acetylation, which was reported to be associated with increased levels of gene expression. Histones interact with the negatively charged backbone of the DNA double helix through positively charged lysine residues. When these residues are acetylated, the histone disassociates from the DNA, and so the DNA itself becomes uncoiled. Histone acetyltransferases (HAT's) transfer the acetyl co-enzyme A to the lysine residue, neutralizing the positive charge. This loosens chromatin packaging and correlates with transcriptional activation. In addition, acetylated histones can also recruit transcriptional activators containing an acetyl-lysine binding module, known as the “Bromo” domain. Histone deacetylases (HDAC's) remove the acetyl groups, thus re-establishing the positive charge in the histones, leading to increased DNA binding and repression of transcription. HDAC inhibitors such as trichostatin A and suberoylanilide hydroxamic acid (SAHA) have demonstrated a number of cancer-fighting properties in cultured cells, such as induction of growth arrest, differentiation, and apoptosis (Meehan et al., 2004).

In addition to acetylation, histones can be modified by methylation, phosphorylation and mono-ubiquitylation. The effects of methylation are not as predictable as acetylation. For example, H3 K4 (the fourth lysine residue of histone type H3) methylation is indicative of transcriptional activation, while H3 K9 or K27 methylation seems to have an opposite, repressive effect. Moreover, each lysine residue can accept from one to three methyl groups, adding an additional layer of complexity (Sarg et al., 2002). Enzymes in the histone lysine methyltransferase family have a conserved functional domain known as the SET domain (Schneider et al., 2002). Deregulation of this domain has been implicated in the development of cancer.

Phosphorylation events on some H2 subtypes of histones can lead instead to apoptosis, the process by which a cell kills itself in response to extracellular signals associated with development, age, or damage to the cell's DNA (Ajiro, 2000). Cancer can be caused by a failure of a genetically damaged cell to undergo apoptosis When a cell divides, new histones are synthesized, mostly during S-phase (Verreault, 2000). A cell in S-phase will therefore have about twice the number of histones of a non-proliferating cell. In addition, histones H3 and H4 are acetylated prior to being deposited at the DNA replication fork, after which they are rapidly deacetylated (Verreault, 2000). This rapid deacetylation is necessary to prevent errors in chromosome segregation during mitosis (Verreault, 2003). Thus, both the number and post-translational modifications of histones change during the uncontrolled cellular proliferation associated with pre-neoplasia and neoplasia. Therefore, in certain embodiments, the present invention provides methods for predicting the recurrence of cancer, the progression of cancer, or the survival of a cancer patient that comprise assessing histone levels and/or histone modification.

Those of ordinary skill in the art will be familiar with a number of ways to assay histones and histone post-translational modifications. For example, because methylation and acetylation are covalent modifications, common protein biochemical techniques that separate proteins based on size such as acrylamide gels can help to differentiate histone proteins with different degrees of modifications (Gui et al., 2004). With knowledge of histone amino acid sequence, the use of endoproteases to cleave the histones at specific sites followed by separation can further specify which region of the histone is modified. (Koutzamani, 2002) Differently methylated histones can be separated and analyzed using reverse-phase high performance liquid chromatography (RP-HPLC) (Sarg, 2002). Digestion with specific endoproteases such as Glu-C or Lys-C allows for easier analysis of the regions of interest following standard protein separation techniques (Sarg, 2002). HDAC activity can be assayed by combining an HDAC-containing sample with radioactively labeled histones, then incubating some samples with 250 mM sodium butyrate, which inhibits HDAC (Meehan, 2004). Rundquist has taken advantage of the salt-dependent disassociation of H1 from chromatin to establish a fluorochrome assay that measures the affinity of linker histones for chromatin (Koutzamani, 2002). In addition, differently modified histones present different targets for antibodies, many of which are commercially available. In certain preferred embodiments, histones are assessed using mass spectrometry and in situ techniques described in the preceding sections.

VI. Clinical Implications

Embodiments of the present invention provide methods for the detection of cancerous and pre-cancerous cells in tissues. In particular embodiments, the present invention provides methods for the detection of field cancerization in a patient undergoing tumor resection. Information obtained by these methods will improve the ability of physicians to treat cancer patients. Accordingly, in certain embodiments of the invention, the methods comprise administering an anti-cancer therapy to a cancer patient based on the patient's predicted risk of recurrence and/or progression of cancer. For example, a patient identified as having field cancerization may be prescribed further treatment or more aggressive treatment for the cancer. Likewise, a patient identified a being at less risk for cancer recurrence or progression may not require additional anti-cancer therapy beyond surgical resection of the primary tumor or may require a less aggressive treatment, thereby reducing unnecessary exposure to anti-cancer agents. Moreover, the methods of the present invention may comprise modifying a patient's existing anti-cancer treatment based on an analysis by one or more of the methods herein described.

VII. Examples

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### In Situ Proteomics in Artificially Created Tissue Microwells

Introduction. Microdispensing of matrix onto tissue sections for subsequent analysis by MALDI TOF Imaging Mass Spectrometry (IMS) results in improved specificity and limits analyte migration. While molecular fingerprints or images are increasingly used for biomarker discovery and clinical research, they are limited to intact proteins or peptides. An approach is presented here where micrometer sized wells in thin tissue sections are created. These miniaturized reaction vessels are printed in a dense array pattern onto a tissue section. Reagents are then dispensed into the wells and the reaction products detected by IMS. We have studied the formation and properties of these tissue microwells and demonstrate chemical and enzymatic reactions resulting in the identification of tryptic protein fragments directly from a mouse brain tissue section.

Methods. Snap frozen tissues were sectioned in a cryostat, thaw mounted onto conductive ITO glass slides and ethanol washed. An automatic microdispensing platform, previously described for matrix printing, was used to print 100-150 pl sized droplets of acetonitrile/water/TFA 90:10:0.1% v/v onto the tissue section. The resulting wells were characterized using electron- and optical microscopy. Tryptic digestion of the tissue is performed by dispensing buffered solutions of porcine trypsin into the wells. Reactions were carried out in temperature and humidity controlled chambers at 37 ° C. Volatile reagents were removed in a lyophilizer prior to the application of the MALDI matrix consisting of saturated CHCA in acetonitrile/water/TFA 50:50:0.1% v/v. A Voyager DE STR and a 4700 proteomics analyzer from Applied Biosystems operated in linear and reflector mode were used to characterize the reaction products.

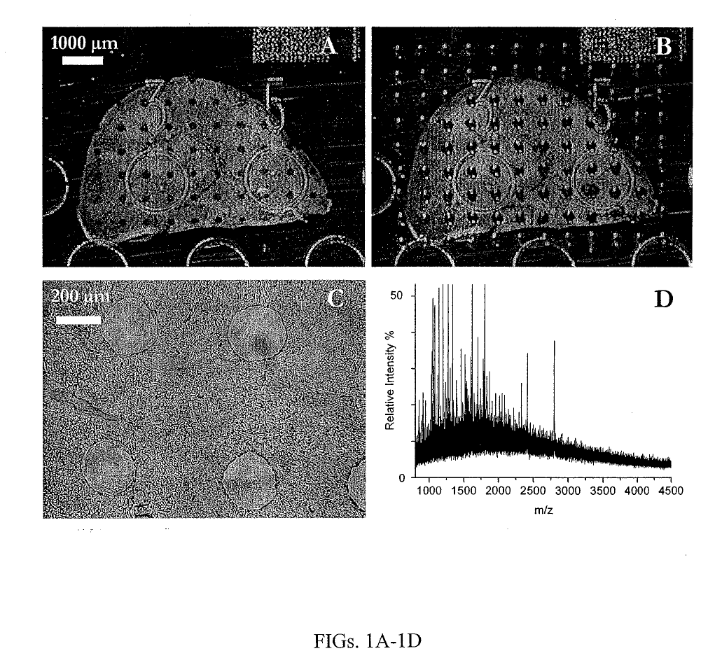

Results. Chemical and enzymatic reactions in thin tissue sections were carried out in situ and reaction products were detected by MALDI TOF MS. Dispensing of organic solvent mixtures in an array onto thin (12-40 μm) tissue sections created 150-400 μm diameter circular microwells. Light microscopy revealed that the cellular structures in the wells were lysed. Stereo pair electron microscopy was used to confirm the well shaped structure. Tissue preparation and composition of organic solvent mixtures was systematically varied to find optimized conditions for the formation of microwells in different tissues such as rat liver and kidney. Arrays of the miniaturized reaction vessels can be printed with up to 40 μm placement accuracy onto regions of interest in a specific sample. Reagents, including trypsin, were dispensed into the wells which served as an anchor for the deposited droplets. Reaction parameters, including buffer composition and reagents, were optimized to ensure compatibility with the MALDI ionization process. Addition of high boiling solvents like 1,2-propanediol was crucial to control the otherwise rapid evaporation of the submicroliter reaction volumes. This new methodology was applied for the identification of proteins from thin tissue sections (FIG. 1). Trypsin was dispensed into microwells on a mouse brain and the resulting complex peptide mixtures were analyzed using tandem MALDI TOF MS followed by Mascot database searching. Several tryptic peptides from proteins, like myelin basic protein (e.g. m/z 1338.7) and the 50 kDa protein beta tubulin (m/z 1052.6 and 1619.8), were identified by MS-MS. Micro reactions carried out in situ followed by molecular detection using mass spectrometry can become a powerful new approach for the spatial mapping and characterization of biologically relevant molecules.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims

VII. References

The following references, to the extent that they provide exemplary procedural or other details supplementary to those set forth herein, are specifically incorporated herein by reference.
K. Ajiro, J. Biol Chem 275, 439-43, 2000.
Bahr et al., J. Mass. Spectrom., 32:1111, 1997.
Bentzley et al., Anal. Chem., 68:2141, 1996.
Braakhuis et al., Cancer Res. 63(8):1727-30, 2003.
Bucknall et al., J. Am. Soc. Mass Spectrometry, 13(9):1015-27, 2002.
Caprioli et al., Anal. Chem., 69:4751, 1997.
Chaurand etal., Anal. Chem., 71:5263, 1999.
Chen et al., J. Chromatogr. B. Biomed. Sci. AppL., 755, 2000.
Desiderio et al., Biopolymers, 40:257, 1996.
Duncan et al., Rapid Commun. Mass Spectrom., 7:1090, 1993.
Eng et al., J. Amer. Soc. Mass Spectrometry, 5:976-989, 1994.
Faulstich et al., Anal. Chem., 69:4349, 1997.
Fenn et al., Science, 246(4926):64-71, 1989.
Gobom et al., Anal. Chem. 72:3320, 2000.
Gui et al., Proc Natl Acad Sci USA, 101(5):1241-6, 2004. (Epub Jan. 20, 2004).
Gygi etal., Mol. Cell. Biol., 19:1720, 1999.
Gygi et al., Nat. Biotechnol., 17:994-999, 1999.
Horak et al., Rapid Commun. Mass Spectrom., 15:241, 2001.
Jespersen et al., Anal. Chem., 71:660, 1999.
Jiang et al., J. Agric. Food Chem., 48:3305, 2000.
Kabarle et al., Anal. Chem. 65(20):972A-986A, 1993.
Kanazawa et al., Biol. Pharm. Bull., 22:339, 1999.
Kazmaieretal., Fres. J Anal. Chem., 361:473, 1998.
Li et al., Trends Biotechnol., 18:151, 2000.
Lovelace et al., J. Chromatogr., 562:573, 1991.
Lynn et al., Rapid Commun. Mass Spectrom., 13:2022, 1999.
Marie et al., Anal. Chem., 72:5106, 2000.
Mason and Liebler, J. Proteome Res., 2(3):265-272, 2003.
Meehan et al., J Biol Chem., 279(2):1562-9, 2004 (Epub Oct. 26, 2003).
Miketova et al., Mol. Biotechnol., 8:249, 1997.
Mirgorodskaya et al, Rapid Commun. Mass Spectrom., 14:1226, 2000.
Muddiman et al., Fres. J Anal. Chem., 354:103, 1996.
Nelson et al., Anal. Chem., 66:1408, 1994.
Nguyen et al., J. Chromatogr., 705:21, 1995.
Norris et al., Anal Chem., 75(23):6642-6647, 2003
Partridge et al., Clin. Cancer Res., 6: 2718-2725, 2000.
Roepstorff et al., Exs., 88:81, 2000.
Sarg et al., J Biol Chem., 18;277(42):39195-201, 2002.
Schneider et al., Trends Biochem Sci 27, 396-402, 2002.
Stoeckli et al., Nat. Med., 7:493, 2001.
Tabor et al., Clin. Cancer Res., 7: 1523-1532, 2001.
Takach et al., J. Protein Chem., 16:363, 1997.
Verreault et al., Genes Dev. 15;14(12):1430-8, 2000.
Villanueva et al., Enzyme Microb. Technol., 29:99, 2001.
Verreault, et al., Med Sci (Paris). 19(12):1181-2, 2003.
Verreault, et al., London Research Institute Scientific Report 2004, 102-103, 2004
Wang et al., J Agric. Food. Chem., 47:1549, 1999.
Wang et al., J Agric. Food. Chem., 47:2009, 1999.
Wang et al., J Agric. Food. Chem., 48:2807, 2000.
Wang et al., J Agric. Food. Chem., 48:3330, 2000.
Wittmann et al., Biotechnol. Bioeng, 72:642, 2001.
Wuetal., J Exp. Med., 185:1681-1691, 1997.
Wuetal.,Anal. Chem., 70:456A, 1998.
Wu et al., Anal Chem., 72:61, 2000.
Yang et al, J Agric. Food. Chem., 48:3990, 2000.
Zaluzec et al., Protein Expr. Purif, 6:109, 1995.
Zhong etal., Clin. Chem. ACTA., 313:147, 2001.
Zhou et al., Nat. Biotechnol., 20:512-515, 2002.
Zlatanova et al., FASEB J., 14(12):1697-704, 2000.
Zweigenbaum et al., Anal. Chem., 71:2294, 1999.
Zweigenbaum et al., Anal. Chem., 74:2446, 2000.

1. A method for predicting recurrence of cancer in a cancer patient undergoing surgical resection comprising:
(a) obtaining a tissue sample from the surgical margin of the resected tumor; and
(b) assessing histone levels in the tissue sample, wherein elevated histone levels in the tissue sample are predictive of the recurrence of cancer.

2. The method of claim 1, wherein the tissue sample is an intact tissue sample.

3. The method of claim 2, wherein assessing histone levels comprises:
(a) subjecting a spatially discrete microregion of the intact tissue sample to one or more physical or chemical treatments; and
(b) assessing the histone levels in a protein sample from the microregion.

4. The method of claim 3, wherein the intact tissue sample comprises at least a second spatially discrete microregion, and the method further comprises subjecting the second spatially discrete microregion to one or more physical or chemical treatments, and assessing histone levels in a protein sample from the second spatially discrete microregion.

5. The method of claim 4, further comprising comparing the histone levels of the first and second spatially discrete microregions.

6. The method of claim 3, wherein the one or more physical or chemical treatments comprise solvent treatment, detergent treatment, lipase treatment, proteolysis, reactive agent treatment, or labeling.

7. The method of claim 6, wherein labeling comprises treatment with an isotope dilution reagent, a labeled antibody, or an enzyme.

8. The method of claim 3, wherein the assessing of histone levels occurs in situ in the microregion.

9. The method of claim 3, wherein the assessing of histone levels occurs after removal of the protein sample from the microregion.

10. The method of claim 4, wherein the first and second spatially discrete microregions receive distinct physical or chemical treatments.

11. The method of claim 4, wherein the first and second spatially discrete microregions receive the same physical or chemical treatment.

12. The method of claim 3, wherein the microregion is a microwell.

13. The method of claim 12, wherein the microwell is between 5 and 200 microns.

14. The method of claim 13, wherein the microwell is between 10 and 100 microns.

15. The method of claim 14, wherein the microwell is about 50 microns.

16. The method of claim 4, wherein the intact tissue comprises at least 6 microregions.

17. The method of claim 4, wherein the intact tissue comprises at least 24 microregions.

18. The method of claim 4, wherein the intact tissue comprises at least 96 microregions.

19. The method of claim 1, wherein assessing the histone levels comprises mass spectrometry.

20. The method of claim 19, wherein the mass spectrometry comprises secondary ion mass spectrometry, laser desorption mass spectrometry, matrix-assisted laser desorption/ionization mass spectrometry, surface-enhanced laser desorption/ionization mass spectrometry, desorption electrospray mass spectrometry, or electrospray mass spectrometry.

21. The method of claim 1, wherein assessing histone levels comprises immunological detection.

22. The method of claim 1, wherein assessing histone levels comprises quantitative detection of RNA.

23. The method of claim 22, wherein the quantitative detection of RNA comprises RT-PCR.

24. The method of claim 22, wherein the quantitative detection of RNA comprises Northern blotting.

25. The method of claim 1, wherein assessing histone levels comprises assessing a histone octamer, a histone unit, or a fragment thereof.

26. The method of claim 25, wherein the histone unit is an H2A, H2B, H3, H4, or Hi.

27. The method of claim 1, wherein the cancer is a cancer of the brain, lung, liver, spleen, kidney, pancreas, small intestine, blood cells, lymph node, colon, breast, endometrium, stomach, prostate, testicle, ovary, skin, head and neck, esophagus, or bone marrow.

28. The method of claim 1, further comprising treating the cancer patient with an anti-cancer therapy.

29. The method of claim 28, wherein the anti-cancer therapy is chemotherapy, radiotherapy, gene therapy, immunotherapy, or hormonal therapy.

30. A method for predicting recurrence of cancer in a cancer patient undergoing surgical resection comprising:
(a) obtaining a tissue sample from the surgical margin of the resected tumor;
(b) subjecting a spatially discrete microregion of the intact tissue sample to one or more physical or chemical treatments; and
(c) analyzing the treated tissue from the microregion for a cancer associated marker or markers using mass spectrometry.

31-49. (canceled)

50. A method for predicting survival of a cancer patient undergoing surgical resection comprising:
(a) obtaining a tissue sample from the surgical margin of the resected tumor; and
(b) (i) assessing histone levels in the tissue sample, wherein elevated histone levels in the tissue sample are indicative of field cancerization or (ii) subjecting a spatially discrete microregion of the intact tissue sample to one or more physical or chemical treatments and analyzing the treated tissue from the microregion for a cancer associated marker using mass spectrometry.

51. A method for predicting cancer progression in a cancer patient undergoing surgical resection comprising:
(a) obtaining a tissue sample from the surgical margin of the resected tumor; and
(b) assessing histone levels in the tissue sample, wherein elevated histone levels in the tissue sample are indicative of field cancerization or (ii) subjecting a spatially discrete microregion of the intact tissue sample to one or more physical or chemical treatments and analyzing the treated tissue from the microregion for a cancer associated marker using mass spectrometry.

52. (canceled)

53. (canceled)

54. A method for detecting field cancerization in a cancer patient comprising:
(a) obtaining a tissue sample from the surgical margin of the resected tumor; and
(b) (i) assessing histone levels in the tissue sample, wherein elevated histone levels in the tissue sample are indicative of field cancerization or (ii) subjecting a spatially discrete microregion of the intact tissue sample to one or more physical or chemical treatments and analyzing the treated tissue from the microregion for a cancer associated marker using mass spectrometry.

55. (canceled)
